(19) United States
(12) Patent Application Publication
(10) Pub. No.: US 2021/0106471 A1
(43) Pub. Date: Apr. 15, 2021

(54) ABSORBENT ARTICLES

(71) Applicant: The Procter & Gamble Company, Cincinnati, OH (US)

(72) Inventors: Yi YUAN, Beijing (CN); Gueltekin ERDEM, Beijing (CN); Mattias SCHMIDT, Idstein (DE); Hui LIU, Beijing (CN)

(21) Appl. No.: 17/068,933

(22) Filed: Oct. 13, 2020

Related U.S. Application Data
(63) Continuation of application No. PCT/CN2019/111207, filed on Oct. 15, 2019.
Application No. PCT/CN2019/111207 is a continuation of application No. PCT/CN2019/124518, filed on Dec. 11, 2019.

(51) Int. Cl. A61F 13/53; A61F 13/49

(52) U.S. Cl. CPC A61F 13/53; A61F 13/49; A61F 2013/530021; A61F 2013/530481; A61F 2013/530014; A61F 2013/530051

(57) ABSTRACT

The present invention relates to a thin, flexible absorbent articles comprising an acquisition-distribution system comprises a first component comprising first thermoplastic fibers and cellulose fibers, the cellulose fibers being less than about 90% by weight of the first component, wherein the absorbent article has an absorption time lower than about 7 sec/g up to a loading range of 100 g, as measured according to Modified Fluid Modified Fluid Acquisition Test; and wherein the absorbent article has a caliper of lower than about 2.5 mm under about 400 g pressure at 2 cm², as measured according to Caliper Test, or a 3-point bending force lower than about 95 g, as measured according to the 3-Point Bending Force Test.

### CROSS REFERENCE TO RELATED APPLICATIONS

This application is a continuation, under 35 U.S.C. § 120, of Chinese PCT Patent Application Serial No. PCT/2019/111207, filed on Oct. 15, 2019, and Chinese PCT Patent Application Serial No. PCT/2019/124518, filed on Dec. 11, 2019, which are both fully incorporated by reference herein.

### FIELD

The invention relates to absorbent articles which are particularly thin, flexible, and comfortable and nevertheless have a high capacity to absorb fluids and are particularly effective in absorbing fluids in a quick manner.

### BACKGROUND

Disposable absorbent articles such as diapers and adult incontinence products are well known in the art. Such disposable articles are designed to absorb and contain body exudates, in particular large quantity of urine. These absorbent articles may comprise several layers providing different functions, for example, a topsheet, a backsheet, an absorbent core disposed between the topsheet and the backsheet, and an acquisition-distribution system disposed between the topsheet and the absorbent core among other layers.

One function of an absorbent core is to absorb and retain the bodily exudates for a prolonged amount of time, for example, overnight for a diaper, minimize re-wet to keep the wearer dry, and avoid soiling of clothes or bed sheets. Some currently marketed absorbent articles comprise absorbent cores comprising an absorbent material which is a blend of comminuted wood pulp (i.e., airfelt) with superabsorbent polymers (SAP) in particulate form, also called absorbent gelling materials. Absorbent articles having a core consisting essentially of SAP as the absorbent material (so called “airfelt-free” cores) have also been proposed.

One function of an acquisition-distribution system (“ADS”) is to quickly acquire liquids or other bodily exudates and distribute them to the absorbent core in an efficient manner. The ADS may comprise one or more layers which may form a unitary structure, or may comprise discrete layers. Some currently marketed absorbent articles comprise an ADS comprising a nonwoven layer and/or a cellulose-containing layer.

There are two specific challenges in delivering thin, flexible, comfort yet highly absorbent articles; the first is having sufficient fluid handling properties such as a high acquisition speed and a mitigated rewet and the second is making the absorbent article thin, flexible and comfortable.

Traditionally, highly absorbent products such as incontinence or diaper products are relatively thick in order to absorb high amounts of discharge delivered quickly. More recently, absorbent articles comprising an airfelt-free core were developed. These absorbent articles have a relatively low caliper and still remain to have a high absorbency but are invariably stiffer and harder. Some thinner products having improved flexibility are hardly able to provide a desired fluid absorbency and a low rewet. These thinner products having a relatively low acquisition speed tend to have a high cuff force to prevent fluid leakage caused by the low acquisition speed which may cause skin markings and irritation when worn by a wearer.

Based on the foregoing, there is a need for absorbent articles which are thin, flexible and are able to sustain absorption speed properties.

Based on the foregoing, there is a need for absorbent articles which are thin, flexible and comfortable, and are able to reduce rewet.

Based on the foregoing, there is a need for absorbent articles which are thin, flexible and are able to allow to stay at a low cuff force range.

### SUMMARY

The present invention relates to an absorbent article comprising a liquid pervious topsheet; a liquid impervious backsheet; an absorbent core disposed between the topsheet and the backsheet; an acquisition-distribution system disposed between the topsheet and the absorbent core, the acquisition-distribution system comprising a first component comprising first thermoplastic fibers and cellulose fibers, the cellulose fibers being less than about 90% by weight of the first component; and a leg gasketing system comprising an inner cuff comprising a cuff sealing disposed in a longitudinally extending matter, and an outer cuff extending transversely outward from the cuff sealing. The absorbent article has an absorption time lower than about 7 sec/g up to a loading range of 100 g, or up to 96 g, as measured according to Modified Fluid Modified Fluid Acquisition Test; and the absorbent article has a caliper of lower than about 2.5 mm under about 400 g pressure at 2 cm², as measured according to Caliper Test, or a 3-point bending force lower than about 95 g, as measured according to the 3-point Bending Force Test.

The article is illustrated in the Figures as a taped diaper. For ease of discussion, the absorbent article and the acquisition-distribution system will be discussed with reference to the numerals referred to in these Figures. The Figures and detailed description should however not be considered limiting the scope of the claims, unless explicitly indicated otherwise, and the invention disclosed herein is also used in a wide variety of absorbent article forms.

### BRIEF DESCRIPTION OF THE DRAWINGS

In the drawings, like numerals or other designations designate like features throughout the views.

FIG. 1 is a schematic plan view of an exemplary absorbent article according to the present invention.

FIG. 2 is a lateral cross-section along 2-2 of the absorbent article of FIG. 1.

FIG. 3 is a cross-section view of an exemplary acquisition-distribution system for an absorbent article according to the present invention.

FIG. 4 is a schematic view of a sample preparation for the Caliper Test.

FIGS. 5A and 5B are schematic views of an example of apparatus for the Caliper Test.

FIG. 6 is a schematic view of a sample preparation for the 3-Point Bending Force Test.

FIGS. 7A-7C are schematic views of an example of apparatus for the 3-Point Bending Force Test.

FIG. 8 illustrates an apparatus used in the Modified Fluid Acquisition Test.

FIG. 9A is a side view of the curved component used in the Modified Fluid Acquisition Test.

FIG. 9B is an end view of the curved component of FIG. 9A.

FIG. 9C is a bottom view of the curved component of FIG. 9A.

FIG. 9D is a bottom perspective view of the curved component of FIG. 9A.

FIG. 9E is a top perspective view of the curved component of FIG. 9A.

FIG. 10A illustrates a top plate assembly used in the Modified Fluid Acquisition Test.

FIG. 10B illustrates equipment used in the Modified Fluid Acquisition Test.

FIG. 11 shows a schematic close up view of an exemplary protrusion having a bulbous shape.

FIG. 12 shows a schematic close up view of an exemplary protrusion having a bulbous shape and a secondary opening at its cap.

### DETAILED DESCRIPTION

Various non-limiting forms of the present disclosure will now be described to provide an overall understanding of the principles of the structure, function, manufacture, and use of an absorbent article according to the present invention. One or more examples of these non-limiting embodiments are illustrated in the accompanying drawings. The features illustrated or described in connection with one non-limiting form may be combined with the features of other non-limiting forms. Such modifications and variations are intended to be included within the scope of the present disclosure.

### Definitions of Terms

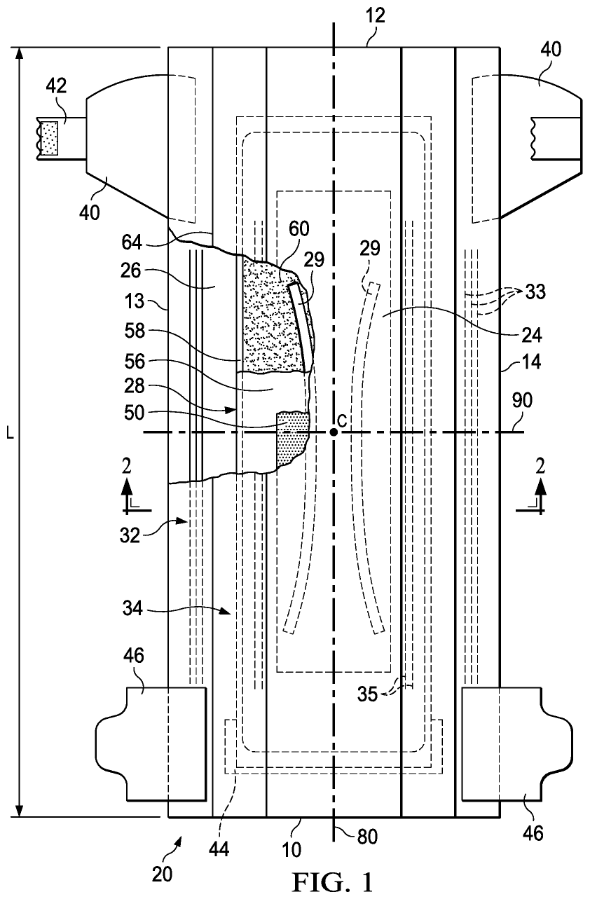

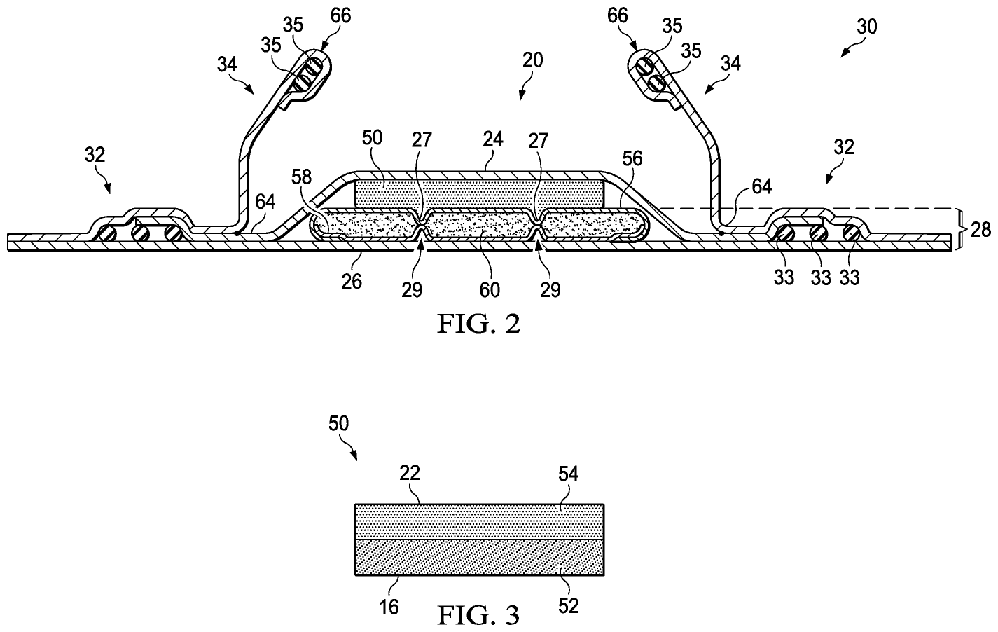

The term “absorbent article” as used herein refers to disposable products such as taped diapers, diapers having a closed waist opening (pants), adult incontinent products, hygiene sanitary napkins and the like, which are placed against or in proximity to the body of the wearer to absorb and contain bodily exudates such as urine, feces or menses discharged from the body. Typical absorbent articles comprise a topsheet, a backsheet, an absorbent core, an acquisition layer and other components. The liquid permeable topsheet forms at least a portion of the wearer-facing side of the article, and the liquid impermeable backsheet forms at least a portion, and typically the whole, of the garment-side of the article. The articles may be provided with fastening elements, such as tapes (taped diapers) or may be provided already pre-formed with a waist opening and a pair of leg openings as in an underwear (pant diapers). The absorbent articles may be for use with babies, infants, women or incontinent adults. Typical features of absorbent articles are further discussed further below, and in relation with the illustrated taped diaper in FIGS. 1 and 2, which is of course for illustration purpose only and not limiting the scope of the inventions, unless specifically indicated otherwise.

The term “air-through bonding” means passing a material such as fibrous web to be bonded through a stream of heated gas, such as air, in which the temperature of the heated gas is above the softening or melting temperature of at least one polymer component of the material being bonding air-through bonding may involve passing a material through a heated oven.

The term “carded web” or “carded nonwoven” means a web or nonwoven comprising staple fibers that are predominantly aligned and oriented in the machine direction using a carding process.

The term “composite” means a structure comprising two or more layers. The two or more layers of a composite may be joined together such that a substantial portion of their common X-Y plane interface.

“Natural fibers” refers to elongated substances produced by plants and animals and includes animal-based fibers and plant-based fibers, as those categories are described herein. Natural fibers, as that term is used herein, include fibers harvested without any post-harvest treatment step as well as those having a post-treatment step, such as, for example, washing, scouring, bleaching.

The term “nonwoven” as used herein refers to a manufactured material, web, sheet or batt of directionally or randomly oriented fibers, bonded by friction, and/or cohesion and/or adhesion, excluding paper and products which are woven, knitted, tufted, stitch-bonded, incorporating binding yarns or filaments, or felted by wet milling, whether or not additionally needled. The fibers may be of natural or man-made origin. The fibers may be staple or continuous filaments or be formed in situ. The porous, fibrous structure of a nonwoven may be configured to be liquid permeable or impermeable, as desired.

The term “staple fiber” means a fiber having a finite length. In general, staple fibers may have a length from about 2 to 200 mm.

### Absorbent Articles

Absorbent articles will now be generally discussed and further illustrated in the form of a baby diaper 20 as exemplarily represented in FIGS. 1 and 2. FIG. 1 is a plan view of the exemplary diaper 20 in a flattened-out configuration with the taped ends opened and the garment-facing side turned up. An article that is presented to the user closed such as a training pant may also be represented flattened out by cutting it along its side waists. This diaper is shown for illustration purpose only as the present invention is applicable to a wide variety of diapers or other absorbent articles. The absorbent article 20 notionally comprises a front edge 10, a back edge 12 and longitudinally-extending first and second longitudinal side edges 13, 14. The front edge 10 forms the edge of the front waist and the back edge 12 of the back waist, which together when worn by the wearer form the opening for the waist of the wearer. The longitudinal side edges 13, 14 can each form one of the leg openings. The absorbent article 20 notionally comprises a longitudinally centerline 80 dividing the article in a left side and a right side, and a perpendicular transversal centerline 90 disposed at half the length of the article as measured on the longitudinal centerline 80, with both centerlines crossing at the center point C. The taped back ends 42 attached on the front of the diaper to such as a landing zone 44.

Referring to FIGS. 1 and 2, the absorbent article 20 includes, from top to bottom, a topsheet 24, an acquisition-distribution system 50, an absorbent core 28 and a backsheet 26. A suitable topsheet may be manufactured from a wide range of materials, such as porous foams, reticulated foams, apertured plastic films, woven materials, nonwoven materials, woven or nonwoven materials of natural fibers (e.g., wood or cotton fibers), synthetic fibers or filaments (e.g., polyester or polypropylene or bicomponent PE/PP fibers or mixtures thereof), or a combination of natural and synthetic fibers.

The absorbent article 20 further comprises a leg gasketing system 30 comprising an inner and outer cuffs 34, 32, which are preferably elasticized with elastic strands 35, 33 respectively. Elasticized back ears 40 having a tape end 42 can be attached to a landing zone 44 at the front of the article. Front ears 46 are typically present in such taped diapers to improve containment and attachment. An outer cover, not indicated in the drawings, may cover at least a portion of, or all of, the backsheet 26 to form a soft garment-facing surface of the absorbent article. The outer cover may be formed of one or multi-layered nonwoven. The nonwoven can comprise a combination of natural fibers and synthetic fibers that are not natural fibers. For example, the nonwoven can comprise both polypropylene fibers and cotton fibers; see, for example, U.S. Patent Application Publication No. U.S. 2017/0203542. The outer cover may be joined to at least a portion of the backsheet 26 through mechanical bonding, adhesive bonding, or other suitable methods of attachment. The backsheet 26 and/or the outer cover may comprise apertures which can promote a breathability conception.

The absorbent article may also comprise other typical components, which are not represented, such as a back-elastic waist feature, a front elastic waist feature, transverse barrier cuff(s), a lotion application, etc.

Absorbent Core

As used herein, the term “absorbent core” refers to a component used or intended to be used in an absorbent article and which comprises an absorbent material and optionally a core wrap. As used herein, the term “absorbent core” does not include the topsheet, the backsheet and any acquisition-distribution layer or multilayer system, which is not integral part of the absorbent core. The absorbent core is typically the component of an absorbent article that has the most absorbent capacity of all the components of the absorbent article. The terms “absorbent core” and “core” are herein used interchangeably.

Referring to FIGS. 1 and 2, the absorbent core 28 can absorb and contain liquid received by the absorbent article and comprise an absorbent material 60, which may be cellulose fibers, a blend of superabsorbent polymers and cellulose fibers, pure superabsorbent polymers, and/or a high internal phase emulsion foam.

The absorbent core 28 may comprise at least one channel 29 free of an absorbent material, or may comprises multiple absorbent material free channels 29 as shown in FIGS. 1 and 2. In an embodiment, the top side 56 of the core wrap may be bonded to the bottom side 58 of the core wrap through the channel 29. The core wrap bonds 27 may at least persist as the absorbent core 28 swells upon liquid absorption and creates three-dimensional channels at the wearer-facing surface of the article. Further details regarding channels are disclosed in WO2015/134359A. Of course, this is entirely optional, the absorbent core may also not have bonded channels, or even unbonded channels.

The absorbent material defines an absorbent material area, which may be rectangular as show in in FIG. 1, but it is also common to have a shaped area which is tapered in the area around the transversal centerline 90. The patent literature is replete with example of such and other components suitable for use in the diapers of the invention, see for example already referred to WO2017/156200A and WO2019/076288A and these will not be discussed in extension herein.

The absorbent material comprises a liquid-absorbent material commonly used in disposable absorbent articles such as comminuted wood pulp, which is generally referred to as airfelt or fluff. Examples of other suitable liquid-absorbent materials include creped cellulose wadding; melt blown polymers, including co-form; chemically stiffened, modified or cross-linked cellulosic fibers; tissue, including tissue wraps and tissue laminates, absorbent foams, absorbent sponges, superabsorbent polymers (herein abbreviated as “SAP”), absorbent gelling materials, or any other known absorbent material or combinations of materials.

The absorbent material in the absorbent core can be any type. For absorbent cores comprising a relatively high proportion of SAP, the SAP content may represent in particular at least 80%, 85%, 90%, 95% and up to 100%, of superabsorbent polymer by weight of the absorbent material. The absorbent material may in particular comprise no or only small amount of cellulose fibers, such as less than 20%, in particular less than 10%, 5% or even 0% of cellulose fibers by weight of the absorbent material. The absorbent core may comprise an absorbent material comprising at least 80%, at least 90%, at least 95%, or at least 99% by weight of the absorbent core. The term “superabsorbent polymer” refers herein to absorbent material, which may be cross-linked polymer, and that can typically absorb at least 10 times their weight of an aqueous 0.9% saline solution as measured using the Centrifuge Retention Capacity (CRC) test (EDANA method WSP 241.2-05E). The SAP may in particular have a CRC value of more than 20 g/g, or more than 24 g/g, or of from 20 to 50 g/g, or from 20 to 40 g/g, or from 24 to 30 g/g. The SAP may be typically in particulate forms (superabsorbent polymer particles), but it not excluded that other forms of SAP may be used such as a superabsorbent polymer foam for example.

Leg Gasketing System

Referring to FIGS. 1 and 2, the absorbent article 20 comprises a leg gasketing system 30 which may be formed by a piece of material which is bonded to the article so it may extend upwards from a wearer-facing surface of the absorbent article and provide improved containment of fluids and other body exudates approximately at the junction of the torso and legs of the wearer. A leg gasketing system 30 may extend from the front edge 10 to the back edge 12. The leg gasketing system 30 comprises an inner cuff 34 comprising a cuff sealing 64 disposed in a longitudinally extending matter, and an outer cuff 32 extending transversely outward from the cuff sealing 64. The inner cuff 34 may be joined at the cuff sealing 64 with the chassis of the article by gluing, fusion bonding, or a combination of other suitable bonding processes. The cuff sealing 64 may be located transversely between the absorbent core 28 and a longitudinal side edge 13, 14. The inner cuff 34 may comprise one, two or more elastic elements 35 close to an inner cuff free edge 66 to provide a better seal. The outer cuff 32 may be joined to chassis of the absorbent article, in particular to the topsheet 24 and/or the backsheet 26 and is placed externally relative to the inner cuff 34. The outer cuff 32 may provide a better seal around the thighs of the wearer. The outer cuff 32 may comprise one or more elastic elements 33 in the chassis of the absorbent article between the topsheet 24 and backsheet 26 in the area of the leg openings.

Acquisition-Distribution System

Referring to FIGS. 1 and 2, the absorbent article 20 of the present disclosure comprises an acquisition-distribution system (“ADS”) 50. One function of ADS 50 is to quickly acquire the body fluid such as urine and distribute them to the absorbent core 28 in an efficient manner. The ADS 50 may comprise a single layer. It may have two or more layers, which may form a unitary structure or may remain as discrete layers which may be attached to each other by, for example, thermal bonding, adhesive bonding or a combination thereof. A unitary structure herein intends to mean that although it may be formed by several sub-layers that have distinct properties and/or compositions from one another, they are somehow intermixed at the boundary region so that, instead of a definite boundary between sub-layers, it would be possible to identify a region where the different sub-layers transition one into the other. Such a unitary structure is typically built by forming the various sub-layers one on top of the other in a continuous manner, for example using air laid or wet laid deposition. Typically, there is no adhesive used between the sub-layers of the unitary material. However, in some cases, adhesives and/or binders can be present although typically in a lower amount that in multilayer materials formed by separate layers.

The ADS according to the present invention comprises a first component comprising first thermoplastic fibers and cellulose fibers. Without being bound by theory, the thermoplastic fibers enhance structural integrity of the fluid distribution layer while also providing for a more open structure. The cellulose fibers provide liquid storage capability and provide a springy open structure that enables quick recovery of the fluid distribution layer to enable readiness for multiple assaults.

The first component comprises the cellulose fibers not higher than about 90%, or from about 50% to about 85%, or from about 60% to about 80%, by weight of the first component. When the amount of cellulose fiber is higher than 90% by weight of the first component may render the first component to collapse when the ADS gets wet, therefore an absorbent speed may be deteriorated. When the amount of cellulose fibers is too low, ADS may not have enough void volume for temporarily storing the fluid initially which may lead to urine leakage. Also, when the cellulose fiber is too low, the ADS may not provide sufficient capillarity to drain fluid from a topsheet.

A wide variety of different cellulose materials may be used for the cellulose fibers. For example, digested cellulose fibers from softwood, hardwood or toon linters can be utilized. Other cellulose fibers include fibers made for regenerated cellulose, polysaccharides or other absorbent fiber-forming compositions. Another example of cellulose fibers is wood pulp e.g., cellulose pulp including treated and untreated pulp such as pulps of hardwood, softwood, straw, chemical pulp, fluff pulp, chemic-mechanical pulp, thermal mechanical pulp and mixtures thereof. Cellulose fibers generally have a fiber length of about from 0.8 to about 10 mm, or about from 2 to 10 mm, or about from 2 to 5 mm.

A wide variety of polymers may be used for the thermoplastic fibers. Examples of suitable fibers include polyolefins such as polypropylene and polyethylene, and copolymers thereof, polyesters such as polyethylene terephthalate (PET), polytrimethyene terephthalate (PTT), and polybutylene terephthalate (PBT), nylons, polystyrenes, copolymer or blends thereof, and other synthetic polymers conventional in the preparation of fibers. The thermoplastic fibers may be staple fibers.

Suitable materials for the thermoplastic fibers include monocomponent or multicomponent fibers, or mixtures thereof. The thermoplastic fiber may comprise a sheath/core bicomponent fiber.

The sheath/core bicomponent fiber may comprises a sheath comprising a polymer having a lower melting temperature than that of a polymer forming the core. The lower melting polymer of the sheath may promote bonding while the higher melting polymer of the core may provide strength to the thermoplastic fiber and thus to the first component. The thermoplastic fibers typically have lengths reigning from about 3-15 mm, or from about 3-10 mm, or from about 3-6 mm. In some embodiments, the sheath/core bicomponent fibers may comprises PE/PET fibers, PE/PP fibers or a mixture thereof In the first component, the thermoplastic fibers may be thermal bonded and may entrapped cellulose fibers.

In some embodiments, the thermoplastic fibers are made by sustainable polymers such as polymers derived from a bio-based material. Sustainable polymers may include polylactic acid and bio-based polyethylene.

The first component may further comprise a binder such as latex. The binder may function as an auxiliary, for immobilize or entrap the cellulose fibers.

The first component may be substantially free of superabsorbent polymer. When a material is substantially free of superabsorbent polymer in the disclosure herein, it intends to mean the material contains superabsorbent polymer less than about 5%, or about 2%, or about 1%, or 0% by the total weight of the material containing superabsorbent polymer. The first component may include superabsorbent polymer. When the first component may include superabsorbent polymer, it may be present in an amount from about 10-35%, or 10-20% by the total weight of the first component.

The first component may have a basis weight in the range of about 20 gsm-140 gsm, or about 30-120 gsm, or about 40-80 gsm. A basis weight of the first component may be determined to balance acquisition-distribution performance and a thickness of the absorbent article.

The first component web may comprise a carded web, air-laid web, wet-laid web, and spunbond web, and the like. In some embodiments, the first component comprises an air-laid web.

Referring to FIG. 3, ADS 50 according to the present invention may further comprise a second component 54 comprising second thermoplastic fibers which is positioned on one surface of the first component 52 in such a way that the first component 52 has an outer surface 16 and the second component 54 has an outer surface 22, and inner surfaces of the first component 52 and the second component are faced to each other. Examples of second thermoplastic fibers include thermoplastic fibers discussed with respect to the first component. The second thermoplastic fiber may or may not be the same as the first thermoplastic fiber. The second component may be free of cellulose fibers.

The second component may have a basis weight in the range of about 20-80 gsm, or about 30-70 gsm, or about 40-60 gam.

In some embodiments, the second component comprises a carded nonwoven. In such embodiments, the second component may comprise an air-through bonding nonwoven. In another embodiment, the second component may comprise spunbond nonwoven or spunbond-meltblown-spunbond (‘SMS”) nonwoven. SMS can mean a three layer, ‘sms’ nonwoven materials, a five layer ssmms' nonwoven materials, or any reasonable variation thereof wherein the lower case letters designate individual layers and the upper case letters designate the compilation of similar, adjacent layers.

An ADS for the present invention may be substantially free of superabsorbent material.

An ADS for the present invention may have a basis weight in the range of about 20-220 gsm, or about 40-160 gsm, or about 20-140 gsm, or about 40-80 gms In some embodiments, the ADS according to the present invention comprises cellulose fibers less than about 40 gsm. A basis weight of the first component may be determined to balance acquisition-distribution performance and a thickness of the absorbent article.

An ADS suitable for absorbent articles according to the present invention may be manufactured via various process known in the industry.

An ADS suitable for the present invention, when consisting of a first component comprising first thermoplastic fibers and cellulose fibers, can be formed for example by air-laying a mixture of the first thermoplastic fibers and cellulose fibers directly onto a carrier wire to form a web, and subsequently subjecting the web to compression and/or thermal treatment in order to bind at least a portion of the first thermoplastic fibers.

When the ADS suitable for the present invention comprises a first component comprising first thermoplastic fibers and cellulose fibers and a second component comprising second thermoplastic fibers, as one example, a web for the ADS may be manufactured by a process comprising the steps of forming a first component web comprising first thermoplastic fibers and cellulose fibers, forming a second component web comprising second thermoplastic fibers, forming a composite web by overlaying the first component web on the second component web or vice versa, and subjecting the composite web to compression, adhesive, and/or thermal treatment in order to bond at least portion of the first and the second thermoplastic fibers.

As another example, a composite wet for the ADS may be manufactured in a continuous process. For example, the process may comprise the steps of a) supplying a second component web comprising second thermoplastic fibers, b) overlaying a first component web comprising first thermoplastic fibers and cellulose fibers on one surface of the second web to form a composite web, and c) subjecting the composite web to compression and/or thermal treatment in order to bond at least portion of the first and the second thermoplastic fibers. In the example, in step a), the second component web may be supplied from a spool on which a previously formed second component web is wounded. Or, the second component web may be supplied by preparing the second component web using a web forming device, for example, a card or spinning beam. The second component web may be supplied using an air-through bonded nonwoven forming device. Step b) may be conducted by air-laid process using at least one forming head. For example, in one or more forming heads a stream of the first thermoplastic fibers and cellulose fibers are homogeneously mixed to form a stream of mixed fibers, and each forming head deposits the stream of mixed fibers on to one surface of the second component web. In step c), the compression may be carried out using one or more pairs of compaction rollers that are disposed following the one or more forming heads. When present, compaction rollers may be heated at a temperature, for example ranging from about 90 to 110° C. In step c), thermal treatment can be conducted using any conventionally known thermal treatment method. Examples of preferable treating process include a thermal treatment apparatus such as a hot air through-type thermal treatment apparatus, a hot air blowing thermal treatment apparatus, an infrared thermal treatment apparatus, or the like. These thermal treatment apparatuses are typically provided with a conveying support for supporting and conveying a web. In one embodiment, the thermal treatment may be carried out by transporting the air-laid web to a heating oven which is maintained at a temperature that is sufficient to soften and melt at least portion of the first and/or the second thermoplastic fibers. For example, in embodiments where the composite web comprises a sheath/core bicomponent fiber, the composite web may be heated to a temperature above the melting point of the sheath but below the melting point of the core so that the sheath component is sufficiently melt and/or soften, and bond at a point of contact or intersection of the fibers.

An ADS suitable for the present invention, when comprising the first component and a second component, the first component may have a fiber density higher than the second component.

The ADS for the present invention may further comprise one or more additional layers deposited on the outer surface 16 of the first component 52.

The absorbent article of the present invention comprises a liquid pervious topsheet, a liquid impervious backsheet, an absorbent core disposed between the topsheet and the backsheet, and an ADS disclosed herein disposed between the topsheet and the absorbent core.

Components of the disposable absorbent article described in this specification can at least partially be comprised of bio-sourced content as described in US 2007/0219521A1 Hird et al published on Sep. 20, 2007, US 2011/0139658A1 Hird et al published on Jun. 16, 2011, US 2011/0139657A1 Hird et al published on Jun. 16, 2011, US 2011/0152812A1 Hird et al published on Jun. 23, 2011, US 2011/0139662A1 Hird et al published on Jun. 16, 2011, and US 2011/0139659A1 Hird et al published on Jun. 16, 2011. These components include, but are not limited to, topsheet nonwovens, backsheet films, backsheet nonwovens, side panel nonwovens, barrier leg cuff nonwovens, super absorbent, nonwoven acquisition layers, core wrap nonwovens, adhesives, fastener hooks, and fastener landing zone nonwovens and film bases. In at least one embodiment, a disposable absorbent article component comprises a bio-based content value from about 10% to about 100% using ASTM D6866-10, method B, in another embodiment, from about 25% to about 75%, and in yet another embodiment, from about 50% to about 60% using ASTM D6866-10, method B. In order to apply the methodology of ASTM D6866-10 to determine the bio-based content of any disposable absorbent article component, a representative sample of the disposable absorbent article component must be obtained for testing. In at least one embodiment, the disposable absorbent article component can be ground into particulates less than about 20 mesh using known grinding methods (e.g., Wiley® mill), and a representative sample of suitable mass taken from the randomly mixed particles.

In some embodiments, the absorbent article of the present invention comprises at least one of a topsheet and a backsheet comprising a natural fiber.

In some embodiments, the absorbent article of the present invention comprises an ADS disclosed herein wherein the first component has a fiber density higher than the second component.

When the ADS of the present invention comprises the first component and the second component, the ADS is disposed in the absorbent article in such a way that the second component is disposed between the topsheet and the first component.

In one embodiment, the absorbent article of the present invention comprises an absorbent core comprising at least about 80%, about 85%, about 90% or about 95% of superabsorbent polymers by weight of the absorbent core.

The absorbent article according to the present invention has a low caliper and preferable flexibility, but still have a desirable fluid handling property. The absorbent article according to the present invention has an absorption time lower than about 7 sec/g, or lower than about 6.5 sec/g, or lower than 6 sec/g, or even lower than 5.5 sec/g up to 100 g, or up to 96 g loading amount as measured according to Modified Fluid Modified Fluid Acquisition Test. It also has a caliper of lower than about 2.5 mm, or lower than about 2.3 mm, or lower than about 2.0 mm under 400 g pressure at 2 cm² as measured according to Caliper Test; and/or has a 3-point bending lower than about 95 g, or lower than about 90 g, or lower than about 85 g, or lower than about 80 g, or lower than 75 g as measured according to 3-point Bending Force Test.

Flexibility of absorbent articles such as diapers may be demonstrated using a simple method below.
1) Place an equipment comprising a metal stand which two standing poles distanced away, adjustable according to the length of sample absorbent article; a metal bar each side of which is hold by each of the standing poles; and a torque wrench rotatably connected to the metal bar. Two clips are attached to the metal bar so that a test absorbent article can be fixed by the clips.
2) Set the torque wrench to a predetermined torque value.
3) Open a diaper. In the case of a pants diaper, tearing side-seams to open the diaper.
4) Fix a front end of the diaper to one clip and a rear end of the diaper to the other clip.
5) Start manually rotating the torque wrench.
6) Record the cycling rounds of the torque wrench until the predetermined torque is reached. The torque wrench will “click” when the predetermined torque is reached. The method can be modified as below.
1) Place an equipment described above.
2) Open a diaper. In the case of a pants diaper, tearing side-seams to open the diaper.
3) Fix a front end of the diaper to one clip and a rear end of the diaper to the other clip.
4) Start manually rotating the torque wrench for predetermined rounds.
5) Record the torque when the predetermined rounds are completed.

The absorbent article of the present invention may have a rewet less than about 120 mg, or less than about 110 mg, or less than about 105 mg, or even less than about 100 mg as measured according to Collagen Rewet Test.

The absorbent article of the present invention may have a caliper of lower than about 9 mm, or lower than about 8 mm, or lower than about 7 mm under 3 g pressure at 2 cm² as measured according to Caliper Test.

The absorbent article of the present invention is thin and flexible, and is able to sustain fluid handling properties such as absorption speed properties and/or rewet.

The absorbent article of the present invention, despite its thin caliper, may have a low cuff force providing conform during wearing and avoiding skin markings and irritation when worn by a wearer without a fluid leakage risk. The absorbent article of the present invention comprises an inner cuff and an outer cuff and at least one of the inner cuff and the outer cuff may have a cuff force lower than about 0.3N, or lower than about 0.25N, as measured according to Cuff Force Test. In some embodiments, the absorbent article of the present invention comprises an inner cuff having a cuff force lower than about 0.3N, or lower than about 0.25N, as measured according to Cuff Force Test. In the embodiments, the absorbent article of the present invention may comprise an inner cuff and an outer cuff both of which have a cuff force lower than about 0.3N, or lower than about 0.25N, as measured according to Cuff Force Test.

In one embodiment, the absorbent article of the present invention does not have a layer comprising cellulose fibers except the ADS especially between a topsheet the absorbent core.

In another embodiment the absorbent article of the present invention comprises at least one of components such as a topsheet, an absorbent core, an ADS and a backsheet of the absorbent article comprises natural fibers.

Absorbent articles of the present disclosure may be “devoid of” or “free of” particular undesirable materials, ingredients, or characteristics in some forms. “Devoid of,” “free of,” and the like, as those terms are used herein, can mean the absorbent article does not have more than trace amounts of background levels of the material, ingredient, or characteristic following these qualifiers; the amount of the material or ingredient does not cause harm or irritation that consumers typically associate with the material or ingredient; or the material or ingredient was not added to the absorbent article intentionally. In some instances, “devoid of” and “free of” can mean there is no measurable amount of the material or ingredient. For example, the absorbent article in some forms contain no measurable amounts of chlorine—that is, the article is characterized as being totally chlorine free.

The ADS according to the present invention may be mechanically deformed. The ADS may comprise a plurality of protrusions extending outwardly from at least one surface of the ADS and the protrusions extending outwardly are oriented towards the absorbent core of the absorbent article. Deformed ADS may improve its mechanical properties such as flexibility and cushiness which are considered trade-offs.

Absorbent Article Manufacturing Process

The absorbent articles of the invention may be made by any conventional methods known in the art. In particular the articles may be hand-made or industrially produced at high speed. Typically, adjacent layers and components will be joined together using conventional bonding method such as adhesive coating via slot coating or spraying on the whole or part of the surface of the layer, or thermo-bonding, or pressure bonding or combinations thereof. Other glues or attachments are not represented for clarity and readability but typical bonding between the layers of the article should be considered to be present unless specifically excluded. Adhesives may be typically used to improve the adhesion of the different layers, for example between the backsheet and the core wrap. The glues used may be any standard hotmelt glue as known in the art.

In one embodiment, a process for producing an absorbent article of the present invention comprises: a) supplying a topsheet material having a wearer-facing surface and a garment-facing side, b) supplying an absorbent core to the garment-facing side of a topsheet material, the absorbent core having a wearer-facing surface and a garment-facing side, and c) supplying a backsheet material to the garment-facing side of the absorbent core, the backsheet material comprising a wetness indicator, wherein the absorbent core comprises an absorbent material and a core wrap at least partially covering the absorbent material, the core wrap comprising a nonwoven web of the present invention.

Mechanical Deformation

ADS disclosed herein may be mechanically deformed by a conventional mechanical deformation process in order to improve its mechanical properties.

Generally, the deformed ADS is made by a method comprising the steps of: a) providing at least one ADS material; b) providing an apparatus comprising a pair of forming members comprising a first forming member (e.g. a “male” forming member) and a second forming member (e.g. a “female” forming member); and c) placing the ADS material(s) between the forming members and mechanically deforming the ADS material(s) with the forming members. The forming members have a machine direction (MD) orientation and a cross-machine direction (CD) orientation. The first and second forming members can be plates, rolls, belts, or any other suitable types of forming members. The mechanical deformation typically includes passing the ADS material between two rolls having a specific intermeshing pattern on their surfaces.

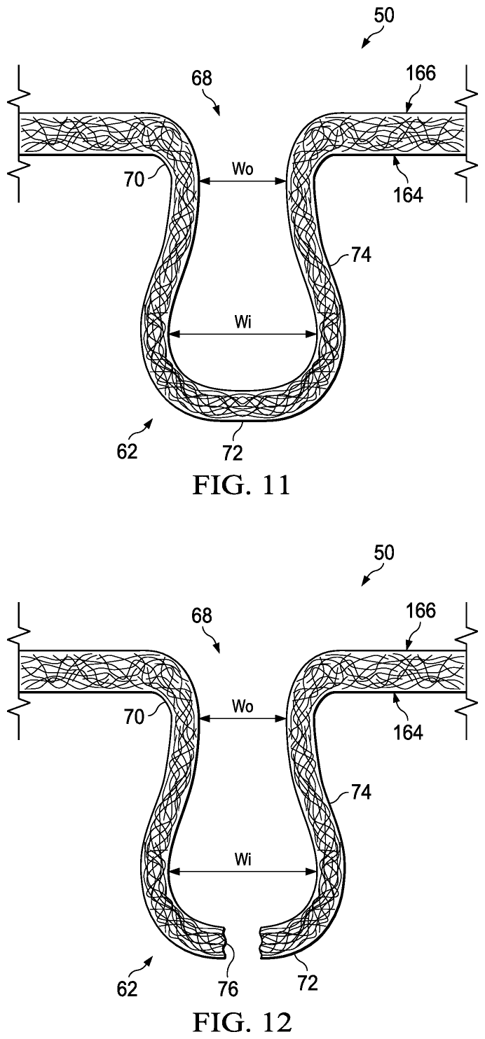

As a result of the mechanical deformation, referring to FIG. 11, a plurality of protrusions 62 extending outwardly from a first surface 164, of the ADS 50 are formed by displacing fibers of the ADS material away from the first surface 164. Simultaneously, openings 68 corresponding to the protrusions are formed in the second surface 166 of the nonwoven. The plurality of protrusions 62 thus formed are preferably discrete protrusions. The mechanical deformation process is different from a conventional embossing process where fibers are compressed inwardly and do not form outwardly extending protrusions.

Various apparatus and methods for making such three-dimensional protrusions have been disclosed in the art. U.S. Pat. No. 8,502,013 (Zhao et al., P&G) for example illustrates in FIG. 6 and following a pair of inter-meshing rolls for a process commonly referred to a SELF process, that may be used to deform a precursor nonwoven. FIG. 14 and following of this reference illustrate a rotary knife apparatus (RKA) and process that uses pointed, pyramidal-shaped teeth on one roll which may provide secondary opening at the tip of the protrusions.

WO2016/040101A1 (Strube et al., P&G) discloses a nonwoven deformation process (referred to as nested-SELF process) to make a nonwoven having discrete three-dimensional bulbous protrusions with wide base openings. In this process, with an exemplary apparatus is shown on a perspective view FIG. 21 and following of WO2016/040101A1, the first and second forming members are in the form of non-deformable, meshing, counter-rotating rolls that form a nip therebetween. The precursor nonwoven is fed into the nip between the rolls. Although the space between the rolls is described herein as a nip, as discussed in greater detail in WO2016/040101A1, in some cases, it may be desirable to avoid compressing the precursor nonwovens(s) to the extent possible. This nested-SELF process is advantageous as it provides bulbous protrusions. These protrusions 62 comprise a base 70 proximate the first surface 164 of the ADS 50, an opposed distal end extending outward in the Z-direction from the base 70, side walls 74 between the base and the distal end of the protrusion 62, and a cap 72 comprising at least a portion of the side walls and the distal end of the protrusions. The side walls 74 have interior surfaces, wherein the interior surfaces of the side walls define a base opening 68 at the base of the protrusion, wherein said cap 72 has a portion with a maximum interior width Wi, and the base opening 68 has a width Wo, wherein the maximum interior width Wi of the cap 72 of the protrusion is greater than the width Wo of the base opening 68. These bulbous protrusions are particularly resilient and can e.g. typically at least partially recover their shape or at least not collapse under compression in the package of absorbent article.

The processes may also be conducted such that one or more secondary openings 76 are formed at the cap 72 or the distal end of the protrusions, due to the fibers of the ADS material not being sufficiently elongatable and breaking at the tip of the protrusions as illustrated in FIG. 12. This may be desired so that a fluid can more quickly move through the ADS towards the absorbent core. When an ADS comprising a multi-layer material is deformed, the second opening 76 may extend through all the layers of the material, or only be formed in one layer, for example the one forming the external surface of the deformed ADS 50.

Another related mechanical deformation process that may be used to make deformed an ADS is disclosed in WO2012/148,944 (Marinelli et al., P&G), where two rolls having intermeshing male elements are used to deform the nonwoven web. This is process may be referred to as SELF-on-SELF (SoS) and is illustrated on FIG. 16 and following of WO2012/148,944A1. The deformed ADS obtained by such a process may comprises protrusions formed on a first surface of an ADS and equivalent protrusion formed on the second surface of the ADS, with the corresponding openings. The SoS process can be used on a single layer material or dual or multi-layer material.

Generally, the protrusions may be uniformly distributed on the forming members and thus on the deformed ADS. The protrusions may also be distributed according to a pre-determined pattern by arranging the male and/or female elements on the forming elements, in particular forming rolls, according to a desired pattern. The average number of protrusions on the deformed ADS may typically range from 0.5 to 5 per square centimeter.

### Measurements

Unless particularly mentioned, all tests are performed in a room maintained at 23±2° C. and 50±5% relative humidity, and samples are pre-conditioned in a room maintained at about 23±2° C. and about 50±5% relative humidity, for at least 2 hours prior to testing.

1. Caliper Test

A caliper of a sample is measured using an Electronic Tensile Tester with a computer interface such as the MTS Criterion C42 running TestWorks 4 Software (available from MTS SYSTEMS (CHINA) CO., LTD) or equivalent instrument. A load cell is selected so that force results for the samples tested will be between 10 and 90% of capacity of the load cell used.

Sample Preparation

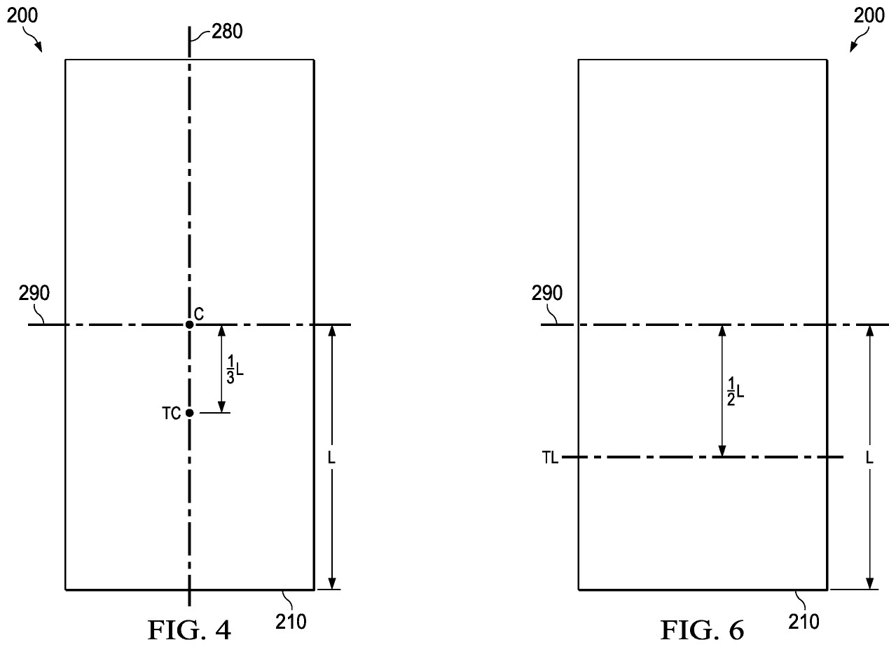

Remove cuff materials from an absorbent article. Also remove the front and back ears of the article, or in the case of a pant, the belt or side panels. Referring to FIG. 4, lay the cuff-removed absorbent article 200 on a bench with a topsheet facing upward, and draw a longitudinal centerline 280 and a transversal centerline 290, and mark a center point C where both centerlines cross to each other. Determine a center TC of a test location on the longitudinal centerline 280 which is a point L/3 away from the center point C toward the front edge 210 when L is the distance between the center point C and the front edge 210.

Measurement Procedure

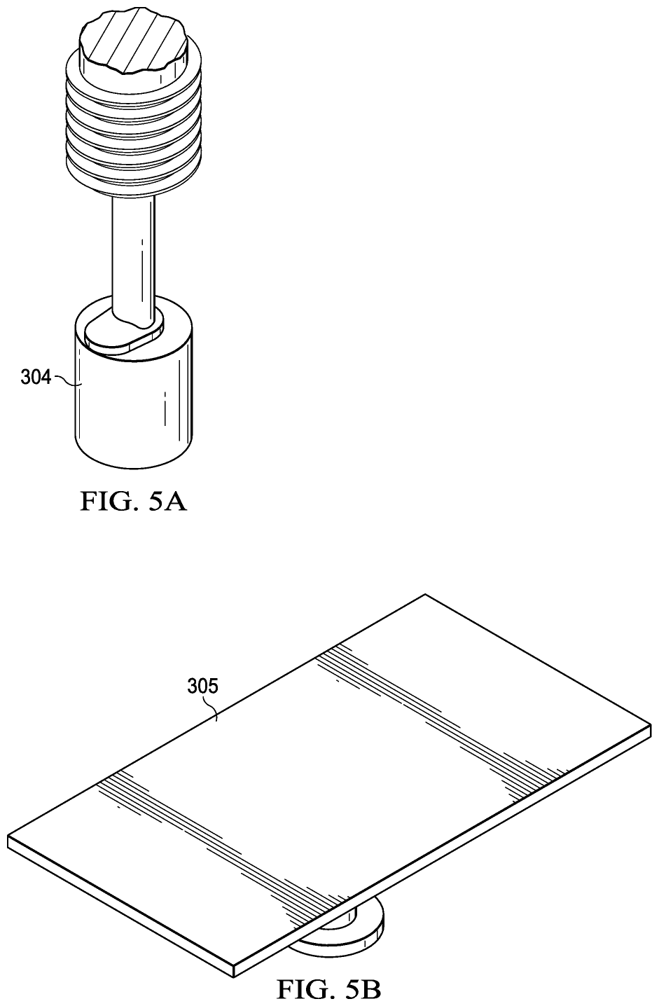

The instrument is calibrated according to the manufacturer's instructions. Referring to FIGS. 5A and 5B, the tensile tester is fitted with a plunger (custom made 200 mm² circular plunger 304) for the upper fixture, and a stage (150 mm×310 mm stage 305) for the lower fixture. The instrument is set up to go through the following steps:

| | | |
|---|---|---|
| Crosshead Speed | 0.2 | mm/sec |
| Pre-load | 1 | gf |
| Final Load Point | 400 | gf |
| Hold Time | 0 | |
| Number of Cycles | 1 | |
| Data Acquisition Rate | 50 | Hz |

The crosshead is drawn down to a position such that the compression plunger 304 is close to, but not touching, the stage 305. Measure the rough thickness of the sample article using ruler. The rough thickness of the sample plus 1 mm is the “platen separation” value. Set the movement of compression plunger 304 to initially reach the compression plate 305, then go up until the predetermined “platen separation” value is provided between the compression plunger 304 and the compression plate 305. Insert sample between the compression plunger 304 and the compression plate 305. Set the crosshead travel to compress the sample article until the load exceeds 400 gf, then return to the “platen separation” position.

Five replicates are analyzed. Calculate and report the caliper under 400 gf as the arithmetic mean of the replicates to the nearest 0.1 mm.
Caliper under 400 gf (Caliper at max pressure-200 gf/cm²: Tm (mm), =interval between stage and the plunger at defined max load=400 gf)

2. 3-Point Bending Force Test

3-point bending force is to measure a bending stiffness of an absorbent region of an absorbent article using for example Texture Analyzer (Stable Micro Systems, UK) or equivalent instrument.

Sample Preparation

Open an absorbent article, and remove cuff materials. Also remove the front and back ears of the article, or in the case of a pant, the belt or side panels. Referring to FIG. 6, lay the cuff-removed absorbent article 200 on a bench with a topsheet facing upward, and draw a transversal centerline 290. Determine a test location line TL which is a line parallel to the transversal centerline 290, and L/2 away from the transversal centerline 90 toward the front edge 210 when the distance between the transversal centerline and C and the front edge 10 is L. Then, cut the cuff-removed absorbent article 200 along the transversal centerline 290 to obtain a test sample.

Measurement Procedure

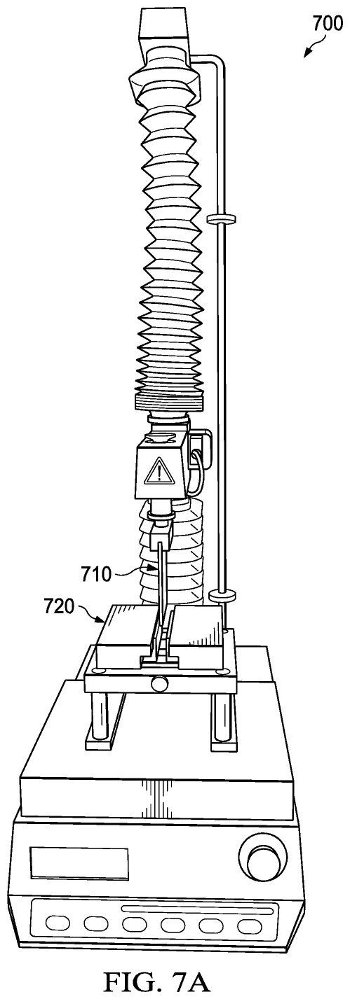

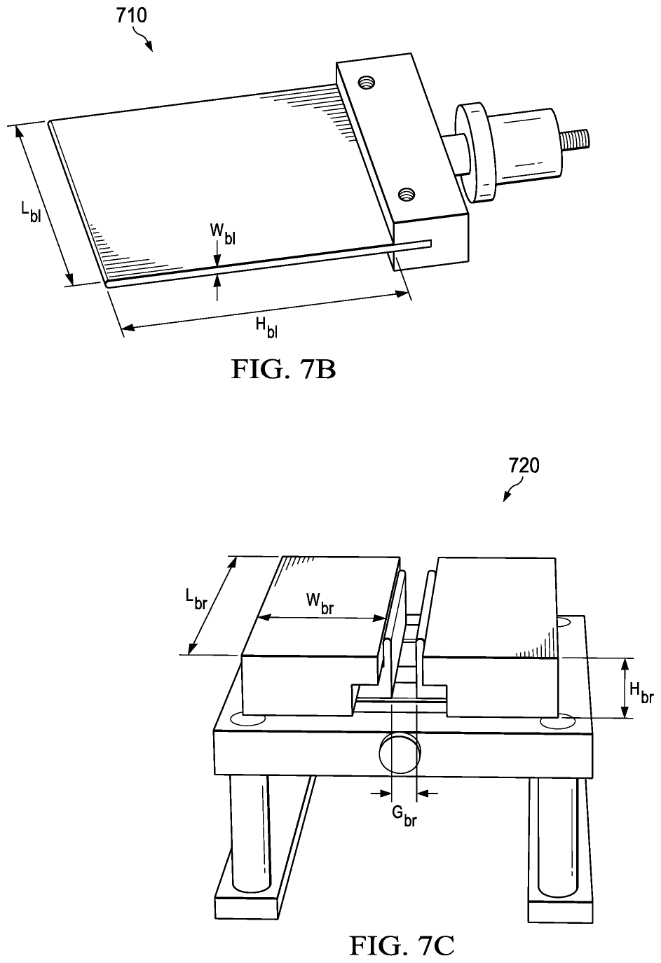

Referring to FIGS. 7A-7C, a Texture Analyzer 700 is fitted with a 3-point bending blade 710 having a 70 mm of a blade length L_{bl}, 3 mm of a blade width W_{bl}, and 80 mm of a blade height H_{bl}, and a bending bridge 720 having 130 mm of a bending bridge length L_{br}, 55 mm of a bending bridge width W_{br}, 25 mm of a bending bridge height H_{br}, and 25 mm of a bending bridge gap G_{br} as shown in FIGS. 7A-7C. The instrument is calibrated according to the manufacturer's instructions. The instrument is set up to go through the following steps:

| Test mode | Compression | |
|---|---|---|
| Test speed | 0.5 | mm/sec |
| Target mode | Distance | |
| Distance | 13 | mm |
| calibrate height setting | 35 | mm |
| Bending bridge gap | 25 | mm |
| Bending bridge depth | 25 | mm |

Put a sample between the bending blade 710 and the bending bridge 720 by placing the sample on top of the bending bridge 720 in such a way that the bending blade 710 is located on top of the test location line TL drawn on the sample. Set the target distance to 13 mm so that the bending blade 710 travels from an initial position and compress the sample then return to the initial position.

Five replicates are analyzed. Calculate and report the 3-point bending force as the arithmetic mean of the replicates to the nearest 0.1 g.

3. Modified Fluid Acquisition Test

The Modified Fluid Acquisition (“MFA”) Test is designed to measure the speed at which 0.9% saline solution is absorbed into an absorbent article that is compressed at 2.07 kPa. A known volume is introduced four times, each successive dose starting five (5) minutes after the previous dose has absorbed. Times needed to absorb each dose are recorded. The test fluid is 0.9% w/v saline solution and is prepared by weighing 9.0 g±0.05 g of NaCl into a weigh boat, transferring it into a 1L volumetric flask, and diluting to volume with de-ionized water.

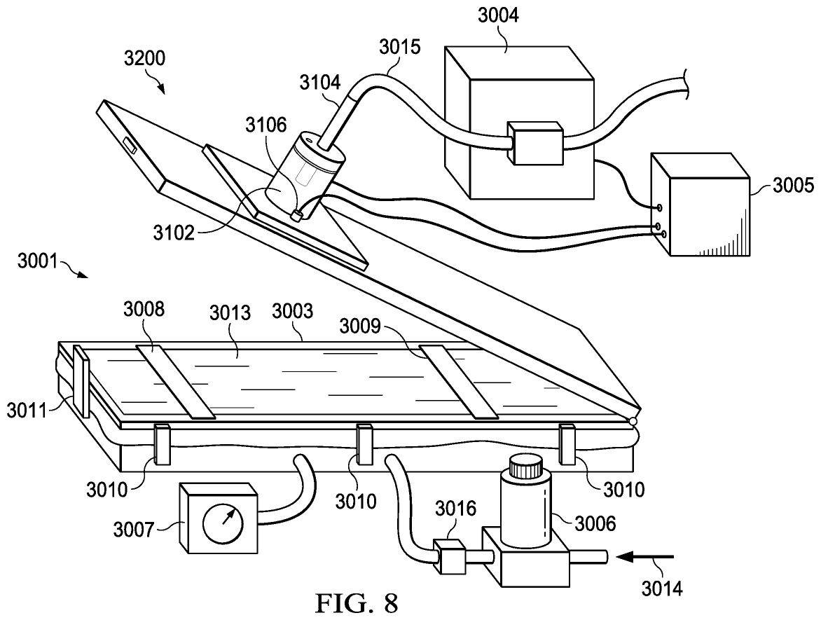

The MFA apparatus is depicted in FIG. 8 through FIG. 10B. The MFA apparatus comprises a bladder assembly 3001 and a top plate assembly 3200 that includes a deposition assembly 3100. A controller 3005 is used to 1) monitor the impedance across electrodes 3106, recording the time interval 0.9% saline solution is in a cylinder 3102, 2) interface with a liquid pump 3004 to start/stop dispensing, and 3) time intervals between dosing. The controller 3005 is capable of recording time events to ±0.01 sec. A house air supply 3014 is connected to a pressure regulator 3006 capable of delivering air at a suitable flow/pressure to maintain 2.07 kPa in the bladder assembly 3001. A liquid pump 3004 (Ismatec MCP-Z gear pump, available from Cole Palmer, Vernon Hills, Ill. or equivalent) capable of delivering a flow of 10-80 mL at a rate of 3-15 mL/s is attached to a stainless steel tube 3104 of the deposition assembly 3100 via tygon tubing 3015.

The bladder assembly 3001 is constructed of 12.7 mm Plexiglas with an overall dimension of 80 cm long by 30 cm wide by 10 cm tall. A manometer 3007 to measure the pressure inside the assembly and a pressure gauge 3006 to regulate the introduction of air into the assembly are installed through two holes through the light side. A bladder 3013 is assembled by draping a 50 mm by 100 mm piece of silicone film, (thickness 0.02″, Shore A durometer value of 20, available as Part #86435K85 from McMaster-Carr, Cleveland, Ohio) over the top of the box with enough slack that the film touches the bottom of the box at its center point. An aluminum frame 3003 with a flange is fitted over the top of the film and secured in place using mechanical clamps 3010. When in place, the assembly should be leak free at a pressure of 3.45 kPa. A front 3008 and back 3009 sample support of 5 cm by 30 cm by 1 mm are used to anchor the sample. The absorbent article is attached to the top surface of the sample supports by either adhesive tape or mechanical “hook” fasteners. These supports can be adjusted along the length of the aluminum frame 3003 via a simple pin and hole system to accommodate different size absorbent articles and to correctly align their loading point.

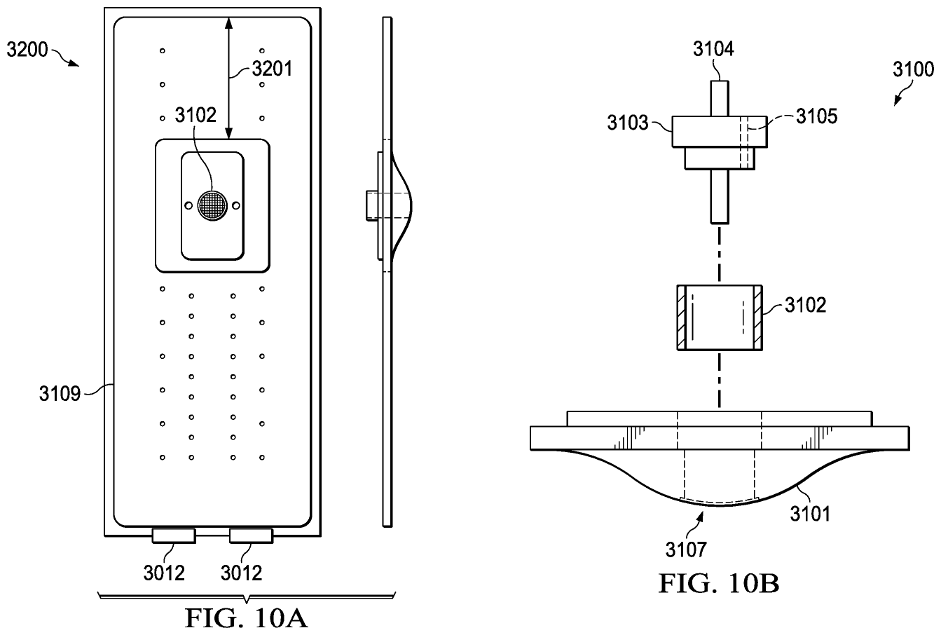

The top plate assembly 3200 is constructed of an 80 cm by 30 cm piece of 12.7 mm Plexiglas reinforced with an aluminum frame 3109 to enhance rigidity. The plate has a cutout 170 mm wide by 201 mm long centered laterally on the plate, 170 mm from the front of the plate 3201 for mounting of the deposition assembly. In addition, the top plate has thirty-six (36) 3.2 mm diameter holes drilled through it distributed as shown in FIG. 10A. The holes prevent air from being trapped under the top plate as the bladder is inflated. The top plate assembly 3200 is connected to the bladder assembly 3001 via two hinges 3012. During use, the top assembly is closed onto the bladder assembly and locked into place using a mechanical clamp 3011.

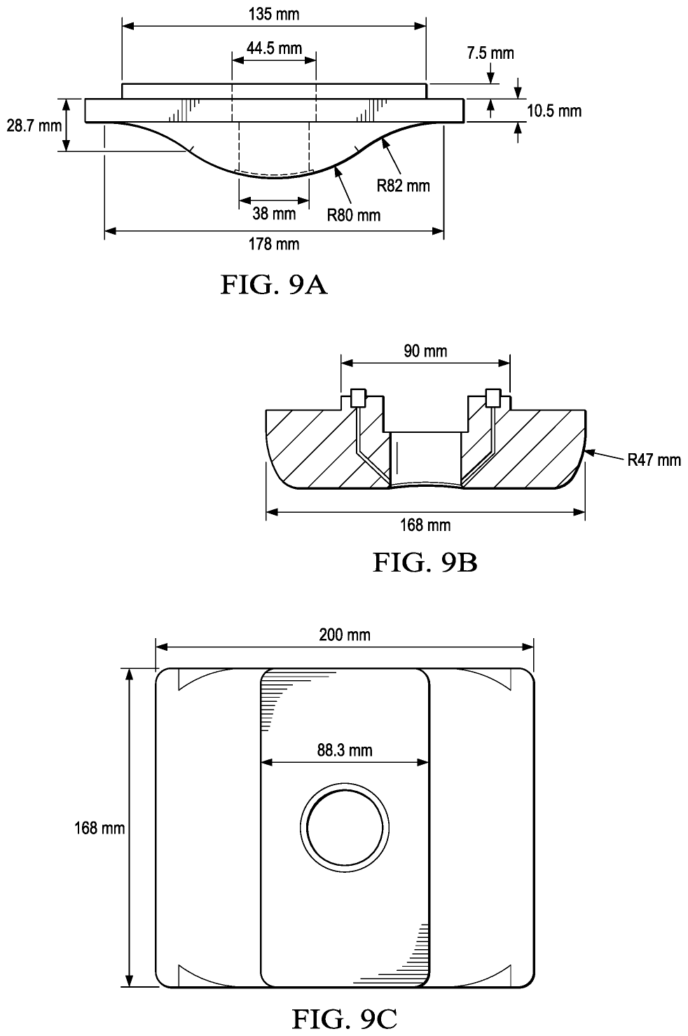

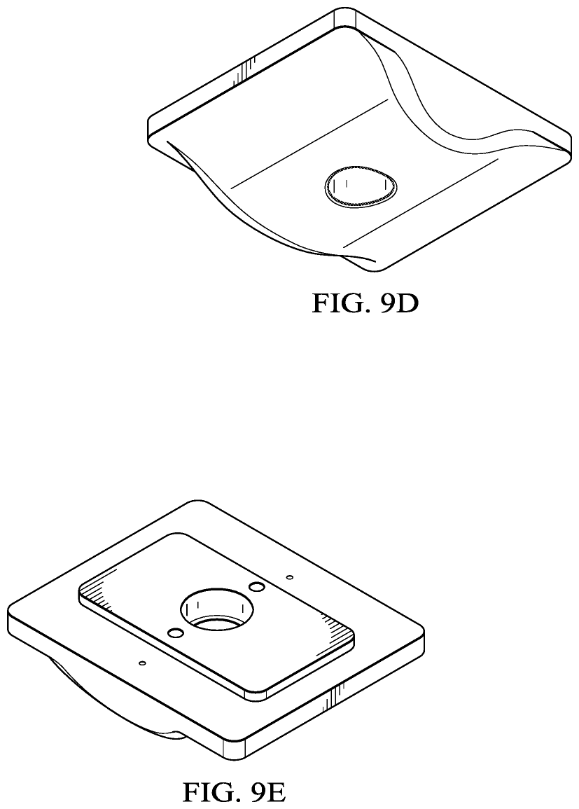

The deposition assembly 3100 is fitted into the top plate 3200 and includes 1) a liquid introduction cylinder 3102, 2) a curved surface 3101 at the loading point of the absorbent article and 3) electrodes 3106 that are used to detect fluid in the cylinder 3102. The detailed dimensions of the curved component are provided in FIG. 9A to FIG. 9E. FIG. 9A is a side view of the curved component. FIG. 9B is an end view of the curved component. FIG. 9C is a bottom view of the curved component. FIG. 9D is a bottom perspective view of the curved component. FIG. 9E is a top perspective view of the curved component. This curved component can be milled or 3D printed. The top portion of the introduction cylinder is a 50.8 mm O.D. Plexiglas cylinder 3102 with a 38.1 mm LD. This is fitted into the curved component to give the introduction cylinder a total height of 100 mm. Imbedded electrodes run from connectors on the upper surface of the curved component and terminate flush with an inside wall of the introduction cylinder, 2 mm from the bottom of the cylinder. The two electrodes are positioned 180 degrees apart. A nylon screen 3107 is cut and affixed flush with the bottom of the cylinder such that the sample cannot swell into the cylinder. A 5 mm semi-circle is cut in the screen in the immediate area of the two electrodes. The deposition assembly is inserted into the top plate as shown in FIG. 10A such that the curved surface is flush with the bottom of the top-plate assembly 3200. The introduction cylinder 3102 is topped with a loose-fitting nylon cap 3103. The cap has a 6.35 mm O.D. steel tube 3104 inserted through its center. When the cap is in place, the bottom of the tube ends 20 mm above the screen 3107. The cap also has an air hole 3105 to ensure negative pressure does not impede the absorption speed.

The absorbent article is first prepared by excising any inner or outer leg cuffs, waist caps, elastic ears or side panels, taking care not to disturb the topsheet that resides above the article's core region. Place the absorbent article flat onto a lab bench and identify the intersection of the longitudinal centerline with the size dependent loading point as defined in Table 1.

TABLE 1
Conditions for Modified Fluid Modified Fluid Acquisition Testing:

| | | Loading Point from | | Single | |
|---|---|---|---|---|---|
| | Approximate | front of Core* | | Rate | Flow |
| Diaper | Baby Weight | Boy | Girl | Volume | Dose |
| Size | Pounds | mm | mm | mL | mL/s |
| 1 | 8 to 13 | 64 | 64 | 24 | 8 |
| 2 | 13 to 17 | 76 | 89 | 24 | 8 |
| 3 | 17 to 28 | 89 | 114 | 50 | 10 |
| 4-6 | 28+ | 102 | 127 | 75 | 15 |

*the boy loading point is used for unisex diapers.

Attach the front end of the absorbent article to the top surface of the front sample plate 3008 by either adhesive tape or mechanical “hook” fasteners with a topsheet facing upward. The placement is such that just the chassis and not the absorptive core overlays the plate. The sample plate 3008 is attached to the aluminum frame 3003 such that the size-dependent Loading Point (as defined in Table 1) of the absorbent article will be centered longitudinally and laterally within the cylinder 3102 when the top plate assembly has been closed. The back end of the absorbent article is secured to the back sample plate 3009 by either adhesive tape or mechanical “hook” fasteners, once again ensuring that only the chassis and not the absorptive core overlays the plate. The back sample plate 3009 is then attached to the aluminum frame 3003 such that the article is taunt but not stretched. The top plate assembly is closed and fastened, and the bladder is inflated to 2.07 kPa±0.07 kPa. The pressure is maintained at this level during the complete loading sequence of the test.

The pump 3004 is primed and then calibrated to deliver the size-dependent volume and flow rate selected from Table 1. Volume and flow rate must be within ±2% of target. The cap 3103 is placed into the cylinder 3102. The controller 3005 is started, which in tum delivers the first dose of 0.9% saline solution. After the volume has been absorbed, the controller waits for 5.0 minutes before addition of the next dose. This cycle is repeated for a total of four doses. If the fluid leaks out of or around the article (i.e., is not absorbed into the article) then the test is aborted. Also if any acquisition time exceeds 1200 seconds, the test is aborted. The acquisition time is defined as the difference between the start time (i.e., when the 0.9% saline is first introduced into the cylinder and that conducting fluid completes the circuit between the electrodes) and the stop time (i.e., when the fluid has completely drained from the cylinder and the circuit between the electrodes is broken). Acquisition times are recorded by the controller for each dose to the nearest 0.01 second. After the last dose is acquired, pressure is applied for an additional 10 minutes. Open the pressure relief valve 3016 to deflate the bladder and then remove the sample from the acquisition system.

In like fashion, run a total of eight (8) replicates for each absorbent article to be evaluated. Calculate and report the Acquisition Times (sec) for each dose as the arithmetic mean of the replicates to the nearest 0.01 sec.

4. Collagen Rewet Test

The Collagen Rewet Test is performed immediately after the MFA Test. The Collagen Rewet Test comprises measuring the mass of fluid expressed from an absorbent article under pressure after loading by the MFA protocol. Collagen sheets are used as the rewet substrate. A suitable collagen is Naturin Coffi collagen sheets (available Naturin GmbH & KG, Germany) or equivalent. Upon receipt, the collagen sheets are stored at about 23° C.±2 C.° and about 50%±2% relative humidity for 2 hours prior to testing. Equipment for the test consists of a Plexiglas disk 70.0 mm in diameter and 20 mm thick and a stainless steel confining weight that rests upon it. The mass of the disk and confining weight combined is 9100 g±2 g which corresponds to a pressure of 23.2 kPa. Collagen sheets are die cut into 70.0 mm diameter circles and stacks of four (4) assembled for use during rewet testing. Measure and record the mass of the dry filter paper stack and record to the nearest 0.0001 g.

Within 30 seconds after the conclusion of the MFA test, remove the absorbent article from the acquisition apparatus and place it flat on a bench top with a topsheet facing upward. Then, place a pre-weighed stack of collagen centered at the loading point (as determined previously in the MFA test), place the Plexiglass disk onto the stack, and gently place the confining weight onto the disk. Wait for 15.0 seconds±0.5 seconds and remove the weight and disc. Immediately measure the mass of the wet filter paper and record to the nearest 0.0001 g. Calculate the Collagen Rewet value as the difference between the wet and dry weight of the stack and record to the nearest 0.1 mg.

In like fashion, run a total of eight (8) replicates for each absorbent article to be evaluated. Calculate and report the Collagen Rewet (mg) for each dose as the arithmetic mean of the replicates to the nearest 0.1 mg.

5. Cuff Force Test

Cuff Force Tests are performed on both the inner and outer cuffs, where present, for an absorbent article. The measurement uses a digital force gauge with a capacity of 0 to 10 N and a minimum resolution of 0.01 N. A suitable gauge is the Quantrol Advanced Force Gauge AFG 10N available from Dillon/Quality Plus Inc, Camarillo, Calif., or equivalent. The sample is mounted using two, a top and a bottom, spring-loaded bar grips. The top grip is hung from the force gauge, with the bottom grip attached to a movable trolley. The force gauge and trolley are mounted on a vertical test stand such that the force gauge is held stationary and the trolley moves vertically along the test stand. The test stand must be tall enough to mount the extended article between the two grips.

Determine the longitudinal length of the inner cuff by taking a representative article and open it, a topsheet facing upward, on a flat surface. If the article is a pant, remove the side panels or belt to open the article flat. Attach a 1000 g±5 g weight to the back waist of the article and hang the article vertically from the front waist. Measure and record the length of the inside cuff structure to the nearest 1 mm. The cuff structure includes the region where elastics are present and the continuous nonwoven that is tacked down to the chassis. This Cuff Length will be used for all replicate articles. Calculate the Maximum Extension Length (MEL) as 0.95×Cuff Length (mm)−25.4 (mm) and record to the nearest 1 mm. Calculate the Final Extension Length (FEL) as 0.85×Cuff Length (mm)−25.4 (mm) and record to the nearest 1 mm. Indicate on the test stand the trolley position corresponding to the MEL (gage from top bar line to bottom bar line of clamps) and the trolley position corresponding to the FEL (gage from top bar line to bottom bar line of clamps).

For each replicate sample, remove, if present, any elastic waist feature. Also remove the front and back ears of the article, or in the case of a pant, the belt or side panels. Remove the inner cuff structure by disengaging any tack down bonds at the front and back waist and then cutting longitudinally, inboard along the base of the cuff. For the outer cuff cut longitudinally, approximately 1 cm inboard of the outer cuff elastic. While excising the cuffs structures, care is taken not to longitudinally stretch the elastics. The cuff specimens are analyzed 15 minutes after removal from the article.

The force gauge is set to continuously take data. Clamp the excised cuff laterally across the specimen, 1.27 mm inboard from the end of the cuff. Zero the force gauge. Move the trolley clamp up the test stand such that the cuff will be in the relaxed state. Attach the other end of the cuff laterally across the specimen, 1.27 mm inboard from the end of the cuff. The specimen should not be twisted within the grips. Begin the measure by lowering the bottom clamp to the MEL position. After one second, raise the bottom clamp till the cuff specimen is in relaxed state. After one second, the bottom clamp is again lowered to the MEL Point. After one second raise the clamp to the FEL position. Wait approximately five seconds then read and record the force from the force gauge to the nearest 0.01 N. Repeat procedure for each inner and outer cuff.

Results are collected for each cuff from 5 replicate articles and averaged keeping corresponding replicates separate. Report as Operator-Side Cuff Force (inner cuff, Drive-Side Inner Cuff, Operator-Side Outer Cuff, Drive-Side and Cuff Force (outer cuff), to the nearest 0.01N.

6. Urine Leakage Test

Urine leakage of an absorbent article is tested by 50 panelists who are caregivers of babies using Size S tape diapers. Each of panels is provided multiple pieces absorbent articles per each test sample. After use of all absorbent articles, the panels evaluate absorbent article leakage condition in the form of yes or no. Urine leakage percentage is calculated as numbers of absorbent articles reported as leakage (yes) divided by the total used pads.

### EXAMPLES

### Example 1-1: Diaper Preparation

Diaper samples 1, 2 and 4 were fabricated using ADS in Table 2 and components of Pampers Hajimeteno Hadaeno Ichiban, size S (Procter and Gamble Japan K.K. Japan) according to Table 3 below. Commercially available diapers were used as diaper samples 3, and 5-8.

TABLE 2

| | ADS 1 | ADS 2 |
|---|---|---|
| Total basis weight | 60 gsm | 80 gsm |
| 1^{st} component | Airlaid nonwoven, 40 gsm | Airlaid nonwoven, 60 gsm |
| | (3% latex, 20% PP/PET | (3% latex, 20% PP/PET |
| | bicopolymer, 77 % | bicopolymer, 77 % cellulose |
| | cellulose fibers) | fibers) |
| 2^{nd} component | 20 gsm, PE/PP carded | 20 gsm, PE/PP carded |
| | air-through | air-through |
| | nonwoven | nonwoven |

TABLE 3

| Sample No. | |
|---|---|
| 1 | Pampers Hajimeteno Hadaeno Ichiban, |
| | size 2 (Procter and Gamble Japan K.K., |
| | Japan) based, replacing a nonwoven |
| | acquisition layer and a cellulose |
| | distribution layer beneath a topsheet with ADS 1* |
| 2 | Pampers Hajimeteno Hadaeno Ichiban, |
| | size 2 (Procter and Gamble Japan K.K. |
| | Japan) based, replacing an acquisition layer |
| | and a distribution layer beneath a |
| | topsheet with ADS 2* |
| 3 | Pampers Hajimeteno Hadaeno Ichiban, |
| | size 2 (Procter and Gamble Japan K.K. |
| | Japan) |
| 4 | Pampers Hajimeteno Hadaeno Ichiban, size 2 |
| | (Procter and Gamble Japan K.K. |
| | Japan) based, removing a cellulose distribution |
| | layer located directly above the |
| | absorbent core |
| 5 | Merries Sarasara Airthrough, |
| | size 2 (Kao Corporation, Japan) |
| 6 | GooN Day Feather, size 2 |
| | (Daio Paper Corporation, Japan) |
| 7 | Lelch Day, size 2 (Dehaa Rossun Infant |
| | Care Product Co., Ltd., China) |
| 8 | Teddy Bear, size 2 (Foshan New |
| | Sensation Sanitary Product Co, Ltd., China) |

*ADS has a width of 90 mm and the same length as an absorbent core.

### Example 1-2: Diaper Properties

Various properties including an absorbent article caliper, a 3-point bending force, an acquisition time, a rewet amount, and cuff tensions of inventive absorbent articles and comparative absorbent articles including several commercially available absorbent articles were tested according to Caliper Test, 3-Point Bending Force Test, Modified Fluid Acquisition Test, Collagen Rewet Test, and Cuff Force Test described in the MEASUREMENT section above, and illustrated in Table 4. Urine leakage was tested according to Urine Leakage Test described in the MEASUREMENT section above with 50 panels and 20 pieces of diapers for each sample.

TABLE 4

| | Invention | | | | | | | |
|---|---|---|---|---|---|---|---|---|
| | samples | | Comparison samples | | | | | |
| Sample No. | 1 | 2 | 3 | 4 | 5 | 6 | 7 | 8 |
| Caliper under | 1.3 | 1.6 | 3.6 | 1.1 | 3.1 | 2.8 | 1.5 | 1.3 |
| 400 gf (mm) | | | | | | | | |
| 3-Point Bending | 53.1 | 70.5 | 145.9 | 48.5 | 77.2 | 99.0 | 63.4 | 52.9 |
| Force (g) | | | | | | | | |
| Acquisition | 3.3 | 3.4 | 1.4 | 7.8 | 3.1 | 2.3 | 8.3 | 7.7 |
| time, up to | | | | | | | | |
| 96 g* (Sec/g) | | | | | | | | |
| Rewet (mg) | 92 | 89 | 73 | 141 | 112 | 117 | 133 | 91 |
| Cuff Force (N), | 0.14 | 0.17 | 0.13 | 0.13 | 0.25 | 0.23 | 0.34 | 0.39 |
| Inner cuff | | | | | | | | |
| Cuff Force (N), | 0.20 | 0.17 | 0.20 | 0.22 | 0.26 | 0.30 | 0.34 | 0.26 |
| Outer cuff | | | | | | | | |
| Leakage* (%) | 1.2 | 1.1 | 0.3 | 1.3 | 1.0 | 1.1 | 1.0 | 1.0 |

*Acquisition time, up to 100 g (Sec/g) was obtained by dividing a total fluid amount by a total acquisition time. Below is Acquisition time, up to 96 g (Sec/g) of Invention sample 1, and Acquisition time (Sec/g) of each sample was obtained in the same way.

| Fluid | Acquisition | Acquisition |
|---|---|---|
| amount | time (Sec) | time (Sec/g) |
| 1^{st} loading: 24 g | 39 | |
| 2^{nd} loading: 24 g | 57 | |
| (Total 48 g) | | |
| 3^{rd} loading: 24 g | 83 | |
| (Total 72 g) | | |
| 4^{th} loading: 24 g | 142 | |
| (Total 96 g) | | |
| Sum (Total | 321 (total | 3.344 (=312/96) |
| loading 96g) | acquisition | |
| | time) | |

It is also important to note that the absorbent article of the present invention is thin and flexible, and even with a low caliper, the absorbent articles of the present invention achieves a fast acquisition time in comparison with Comparison samples 4, 7 and 8 of thin absorbent articles. It is also important to note that the absorbent article of the present, even with a low caliper, achieves about the same or lower level of rewet as a commercially available thick absorbent articles (Comparison samples 5 and 6) as well as thin absorbent articles (Comparison samples 3, 7 and 8). It is also important to note that the absorbent article of the present invention, even with a low caliper, can have a much low cuff force without compromising leakage prevention in comparison with commercially available thick absorbent articles (Comparison samples 5 and 6) as well as thin absorbent articles (Comparison samples 7 and 8).

### Example 2-1: Diaper Preparation

Diaper sample 9 (Silk8 Superflex) was fabricated using ADS 2 in Table 2 above and components of Pampers Ichiban, Pant size L (Procter and Gamble Japan K.K. Japan) according to Table 5 below. Commercially available diapers were used as diaper samples 10 and 11.

TABLE 5

| Sample No. | |
|---|---|
| 9 | Pampers Ichiban, Pant size L (Procter |
| | and Gamble Japan K.K., Japan) based, |
| | replacing a nonwoven acquisition layer |
| | and a cellulose distribution layer |
| | beneath a topsheet with ADS 2 |
| 10 | Pampers Ichiban, Pant size L (Procter and |
| | Gamble Japan K.K., Japan) |
| 11 | Teddy Bear, Pant size L (Foshan New |
| | Sensation Sanitary Product Co, Ltd., |
| | China) |

### Example 2-2: Diaper Properties

Various aspects of diaper samples 9, 10 and 11 were tested by 99 panelists who are caregivers of babies using Size 4 premium pant diapers. Each of panels was provided multiple pieces diapers per each test sample. After use of each diaper, the panels evaluated items below for tested diaper samples which were scored as such: “Poor”=0, “Fair”=25, “Good”=50, “Very Good”=75, and “Excellent”=100. Results are shown in Table 6 below.

TABLE 6

| | Invention | | |
|---|---|---|---|
| RECALL STAGE | sample | Comparison samples | |
| n = 99, 90% CL | Sample 9 | Sample 10 | Sample 11 |
| Overall rating | 68 | 64 | 62 |
| Preventing urine leaks | 80 | 82 | 74 |
| Handling large overnight loads | 76 | 76 | 70 |
| Is breathable/Allowing for | 78 | 73 | 70 |
| air to reach | | | |
| baby skin | | | |
| Keeping baby's skin healthy | 79 | 73 | 74 |
| Being soft overall | 85 | 77 | 78 |
| Being soft against baby's skin | 82 | 77 | 75 |
| Being comfortable for my | 79 | 77 | 74 |
| baby to wear | | | |
| Allowing your child to | 81 | 80 | 76 |
| move around | | | |
| freely | | | |
| The core doesn't bunch | 74 | 71 | 71 |
| in the dry stage | | | |
| The core doesn't | 74 | 70 | 72 |
| bunch/break in the wet | | | |
| stage | | | |

Sample 9 got significantly better score over samples 10 and 11 in most of items reflecting flexibility and comfort as well as leakage prevention.

The dimensions and values disclosed herein are not to be understood as being strictly limited to the exact numerical values recited. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article comprising:
1) a liquid pervious topsheet,
2) a liquid impervious backsheet,
3) an absorbent core disposed between the topsheet and the backsheet,
4) an acquisition-distribution system disposed between the topsheet and the absorbent core, the acquisition-distribution system comprising a first component comprising first thermoplastic fibers and cellulose fibers, the cellulose fibers being less than 90% by weight of the first component, and
5) a leg gasketing system comprising an inner cuff comprising a cuff sealing disposed in a longitudinally extending matter, and an outer cuff extending transversely outward from the cuff sealing,
wherein the absorbent article has an absorption time lower than about 7 sec/g up to a loading range of 100 g, as measured according to Modified Fluid Modified Fluid Acquisition Test, and wherein the absorbent article has a caliper under 400 gf lower than about 2.5 mm, as measured according to the Caliper Test, or a 3-point bending force lower than about 95 g, as measured according to the 3-Point Bending Force Test.

2. The absorbent article according to claim 1, wherein the absorbent core comprises an absorbent material comprising at least 80% superabsorbent polymers by weight of the absorbent material.

3. The absorbent article according to claim 1, wherein the absorbent core is free of cellulose fibers.

4. The absorbent article according to claim 1, wherein the absorbent core comprises at least one channel.

5. The absorbent article according to claim 1, wherein the acquisition-distribution system comprises a second component comprising second thermoplastic fibers, and wherein the second component is located between the topsheet and the first component of the acquisition-distribution system.

6. The absorbent article according to claim 5, wherein the second component is free of cellulose fibers.

7. The absorbent article according to claim 5, wherein the acquisition-distribution system is a unitary structure.

8. The absorbent article according to claim 5, wherein the acquisition-distribution system comprises discrete layers.

9. The absorbent article according to claim 1, wherein the acquisition-distribution system is free of superabsorbent material.

10. The absorbent article according to claim 1, wherein the first component has a basis weight of from about 20 gsm to about 140 gsm.

11. The absorbent article according to claim 1, wherein at least one of the inner cuff and the outer cuff has a cuff force lower than 0.3N, as measured according to Cuff Force Test.

12. The absorbent article according to claim 1, wherein each of the inner cuff and the outer cuff has a cuff force lower than 0.3N, as measured according to Cuff Force Test.

13. The absorbent article according to claim 1, wherein the absorbent article has a rewet lower than 120mg, as measured according to Collagen Rewet Test.

14. The absorbent article according to claim 1, wherein the first component comprises air-laid nonwoven.

15. The absorbent article according to claim 1, wherein the acquisition-distribution system comprises cellulose fibers less than 40 gsm.

16. The absorbent article according to claim 1, wherein the cellulose fibers in the first component comprises wood pulp.

17. The absorbent article according to claim 1, wherein the absorbent article is a diaper or an adult incontinent product.

18. The absorbent article according to claim 1, wherein at least one component of the absorbent article comprises natural fibers.

19. The absorbent article according to claim 1, wherein the acquisition-distribution system is mechanically deformed.

20. The absorbent article according to claim 1, wherein the acquisition-distribution system comprises a plurality of protrusions extending outwardly, and wherein the protrusions extending outwardly are oriented towards the absorbent core of the absorbent article.
